(19) United States
(12) Patent Application Publication
(10) Pub. No.: US 2012/0118299 A1
(43) Pub. Date: May 17, 2012

(54) PATIENT POSITIONING APPARATUS FOR EXAMINATION AND TREATMENT AND METHOD OF THE SAME

(76) Inventor: BROCK MILLER, TORONTO (CA)

(21) Appl. No.: 12/946,047

(22) Filed: Nov. 15, 2010

(51) Int. Cl. A61F 5/37

(52) U.S. Cl. 128/870

(57) ABSTRACT

A patient positioning apparatus for examination and treatment that allows an operator to rotate a patient about three body planes, positioning the semi-circular canals of the patient for proper treatment or diagnosis of conditions caused by abnormalities of the vestibular organs. This apparatus accomplishes three dimensional rotations by rotating about two orthogonal axes of rotation. The efficient and compact design of this apparatus allows the apparatus to be used in clinics or offices too small to accommodate a patient rotation device of the prior art.

### FIELD OF THE INVENTION

The present invention relates generally to a diagnosis and treatment apparatus, and more particularly, relating to an apparatus for controlled spatial positioning of a patient for the diagnosis, treatment, and/or rehabilitation of a medical condition. The apparatus of the present invention is particularly useful for the diagnosis, treatment, and/or rehabilitation of vestibular related conditions.

### BACKGROUND OF THE INVENTION

A considerable number of people experience symptoms of vertigo, dizziness, or other serious debilitating conditions related to balance and the lack of stability. Vertigo and its related conditions are often caused by an imbalance or misplacement of anatomical particles around semi-circular canal systems containing fluids that affect the perception of gravity. The misplacement of these particles can cause an abnormality of the vestibular organs in the inner ear. In a healthy patient, the vestibular organs accurately sense the acceleration of a patient's head and transmit a neurological signal to the central nervous system. The central nervous system will then translate the signal from the vestibular sensory organ into information relating to balance and position of the body. In a patient suffering from vertigo or a related condition, the vestibular organs become displaced from their original position and move into the semi-circular canal system. This displacement causes the vestibular organs to transmit an erroneous signal to the central nervous system, typically causing the patient to perceive a false sense of gravity and rotation.

In the past, most forms of diagnosis and treatment of vertigo and related conditions relied upon instructing a patient to manipulate his or her head into various positions to facilitate the flow of fluids through the semi-circular canals within the inner ear. This manipulation would allow the physician to isolate the specific dislocated vestibular organ or organs causing the abnormality in perception. For treatment, the patient would be instructed to move his or her head in a manner allowing gravity to reposition the vestibular organs outside of the semi-circular canals and back into their normal positions.

Although various machines and devices have been created to rotate a user about three dimensions, many of these devices are manually controlled. In entertainment devices, such as a ride at a carnival, this shortcoming is insignificant since the position of the user does not require a high degree of accuracy. Conversely, when spatial manipulation is used to diagnose and treat a medical condition, a high degree of accuracy and programmability is required to effectively assist physicians to diagnose or treat patients. Of the spatial manipulator systems providing a higher degree of control, the construction of these systems relies on an array of circular or elliptical trusses. Truss based systems not only demand a large footprint for deployment in an office or clinic, but also contain a complicated interconnection of moving parts that restrict the device from effectively rotating a patient about a point aligned with a patient's vestibular organs for proper diagnosis and treatment.

Accordingly, there is a need for an apparatus capable of spatially manipulating a user around point relative to the center of gravity of a patient and in relation to the patient's vestibular organs being diagnosed or treated. Also, there is a need for an apparatus capable of spatially manipulating a user, such that the apparatus is compact in nature so it can easily fit within an office or clinic.

### SUMMARY OF THE INVENTION

The present invention allows the spatial manipulation of a patient about the three body planes, with respect to a patient's semi-circular canals. This invention achieves such rotational freedom by manipulating a patient about two orthogonal axes via the rotation of a connected arm and a connected patient restraint. During rotation of the patient, he or she may be manipulated such that his or her center of gravity is approximately located at the point of intersection between the first and second axes of rotation. This configuration limits the force required to move this patient. Alternately, the patient may be rotated about other center points, such as the organs undergoing treatment by the apparatus. Accordingly, due to the configuration and size of the present invention, it can achieve the required rotational flexibility in a footprint compact enough to fit in a wide variety of spaces otherwise prohibited by a conventional device, such as an office or clinic.

To achieve these and other advantages, in general, in one aspect, a patient spatial movement apparatus for moving a patient through space is provided. The patient spatial movement apparatus includes a base and an arm attached to and supported by the base for rotation about a first axis of rotation. A patient restraint is attached to and supported by the arm for rotation about a second axis of rotation. The patient restraint receives a patient such that the second axis of rotation is at a constant and fixed angle relative to the coronal plane of the patient. The first axis of rotation is normal to the sagittal plane of the patient and the first axis of rotation is orthogonal to the second axis of rotation.

In general, in another aspect, the first axis of rotation and the second axis of rotation intersect about the center of gravity of a patient.

In general, in another aspect, the constant and fixed angle is about 30 degrees.

In general, in another aspect, the invention further includes a controller, a first motor operatively connected to the arm and operable to rotate the arm about the first axis of rotation, and a second motor operatively connected to the patient restraint and operable to rotate the patient restraint about the second axis of rotation. The first and second motors are connected to the controller to receive control signals therefrom to effect the operation of the first motor to rotate the arm about the first axis of rotation and to effect the operation of the second motor to rotate the patient restraint about the second axis of rotation.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The accompanying drawings, which are included to provide further understanding of the invention and are incorporated in and constitute a part of this specification, illustrate preferred embodiments of the invention and together with the description serve to explain the principles of the invention, in which:

FIG. 1 is an isometric view of the patient positioning apparatus constructed in accordance with the principles of the present invention;

FIG. 2 is a side elevation view of the patient positioning apparatus of FIG. 1, showing the base, patient restraint, second axis of rotation, and third axis;

FIG. 3 is a front elevation view of the patient positioning apparatus of FIG. 1, further showing the base, legs, arm, patient restraint, first axis of rotation, and second axis of rotation;

FIG. 4 is a top plan view of the patient positioning apparatus of FIG. 1, further showing illustrating the base, arm, legs, and patient restraint;

FIG. 5 is a side elevation view of the patient positioning apparatus of FIG. 1 with the arm in the zero/home position, further showing the base, legs, arm, patient restraint, first axis of rotation, and second axis of rotation;

FIG. 6 is an isometric view of the patient positioning apparatus of FIG. 1, showing the arm and patient restraint in operation, both rotated 180 degrees;

FIG. 7 is an isometric view of the patient positioning apparatus of FIG. 1, showing or illustrating the patient restraint in operation rotated 180 degrees about the second axis of rotation;

FIG. 8 is an isometric view of an exemplary user input device for use with the patient positioning apparatus of FIG. 1;

FIG. 9 is a block diagram of a controller for the patient positioning apparatus of FIG. 1; and

FIG. 10 is a flow chart of an example program for controlling the patient positioning apparatus of FIG. 1.

### DETAILED DESCRIPTION OF THE INVENTION

As a preliminary matter, it should be noted that in this document (including the claims) directional terms, such as “above”, “below”, “upper”, “lower”, etc., are used for convenience in referring to the accompanying drawings. Additionally, it is to be understood that the various embodiments of the present invention described herein may be utilized in various orientations, such as inclined, inverted, horizontal, vertical, etc., without departing from the principles of the present invention.

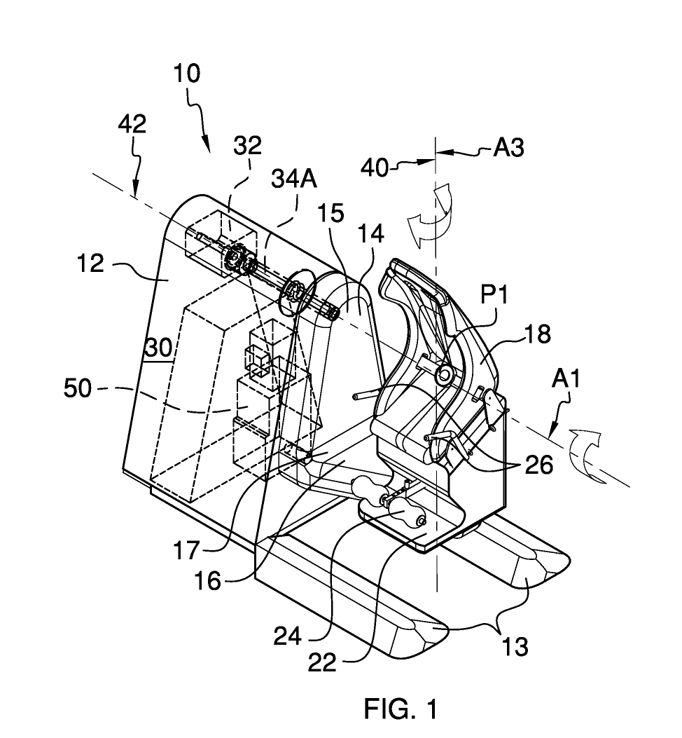

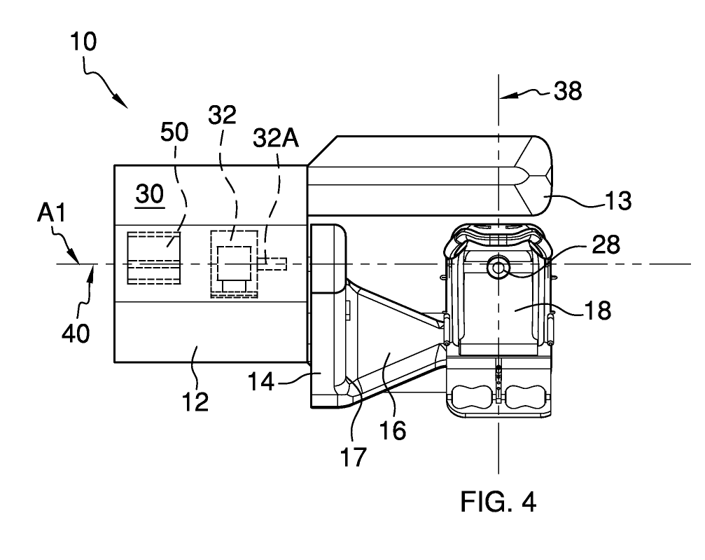

In FIGS. 1-4, there is representatively illustrated an apparatus 10 for use in the diagnosis, treatment, and/or rehabilitation of vestibular conditions. It is important to note here, while the following description may be specific in relation to vestibular conditions, one skilled in the art will appreciate the apparatus could be used for the diagnosis, treatment, and/or rehabilitation of other medical conditions. It will also be appreciated, and while the device is designed for medical use, it could be used in other applications requiring the spatial positioning of a user, for example, such as a patient restraint in a virtual reality environment.

Broadly, apparatus 10 includes a base 12, an arm 14, a patient restraint 18, and a controller 50. Base 12 includes a surface 30, which encompasses base 12 and acts as a cover to isolate and protect the internal components of apparatus 10 from operators or patients. As previously mentioned, apparatus 10 includes a controller 50 that manages the operation of apparatus 10. This controller 50 could be positioned in base 12.

Base 12 acts to stabilize apparatus 10 via the weight and shape of base 12. Base 12 may further include a pair of legs 13 that each extend in the same general direction from about a bottom edge of the base 12 and terminate at or slightly beyond the patient restraint 18. Legs 13 provide enhanced stability for apparatus 10 during both idle and operational states by allowing the weight of apparatus 10 to be more evenly distributed. In one embodiment of apparatus 10, legs 13 may be tapered with the top surface of legs 13 having a more narrow width than the bottom surface of legs 13. This tapering of legs 13 allows for a larger surface area to make contact with the ground surface. Also, tapering of legs 13 may also reduced the risk of an operator unintentionally tripping or stumbling over legs 13.

Still referring to FIGS. 1-4, arm 14 includes a first arm segment 15 and a second arm segment 16 conjoined at elbow 17. First arm segment 15 includes opposed first and second ends 15A and 15B, respectively. Second arm segment 16 includes opposed first and second ends 16A and 16B, respectively. The second end 15B of the first arm segment 15 and the first end 16A of the second arm segment 16 are conjoined at elbow 17. The first arm segment 15 and the second arm segment 16 extend generally normal to each other.

Arm 14 is attached to and supported by base 12 at the first end 15A of the first arm segment 15 for rotation about a first axis of rotation A1, with the first arm segment extending normal to the first axis of rotation A1. In this manner, the second arm segment 16 extends generally parallel to the first axis of rotation A1. Arm 14 is attached to and supported by base 12 for rotation through 360-degrees about the first axis of rotation A1. A first axis motor 32 is operatively connected to arm 14, such as, for example at the first end 15A thereof. Rotational power from the first axis motor 32 is transmitted to the arm 14 causing the arm rotate about the first axis A1 and thus revolving the second arm segment 16 about the first axis of rotation A1. First axis motor 32 can be operated to rotate the arm 14 in both a clockwise direction and a counter-clockwise direction about the first axis of rotation A1.

In an embodiment of apparatus 10, the first axis motor 32 can be located near the bottom of base 12. The low placement of the first axis motor 32 gives the apparatus 10 a lower center of mass, and therefore a larger degree of stability during both idle and operational states. The first axis motor 32 can be operatively connected to arm 14 by any suitable intermediate structure 32A. Examples of intermediate structure 32A include, but are not limited to, chains, belts, gears, drive shafts, or the like and any combinations thereof. Alternatively, the first axis motor 32 can be directly connected to arm 14 in a direct drive relation.

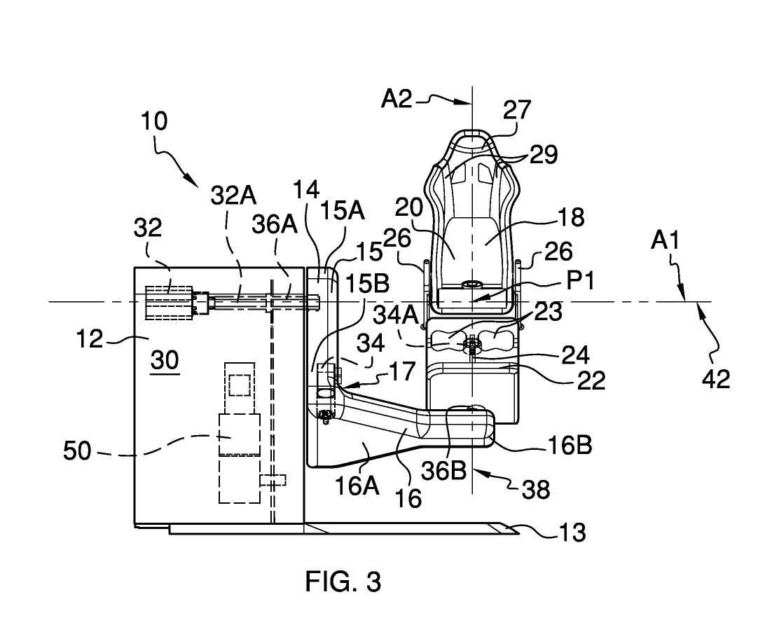

As illustrated in FIGS. 1-4, the patient restraint 18 is attached to and supported by the second arm segment 16, for example at end 16B thereof, for rotation about a second axis of rotation A2 that is normal to the first axis of rotation A1. The patient restraint 18 is attached to and supported by the second arm segment 16 for rotation through 360-degrees about the second axis of rotation A2. A second axis motor 34 is operatively connected to the patient restraint 18 to rotate the patient restraint about the second axis of rotation A2. The second axis motor 34 can be operated to rotate the patient restraint about the second axis of rotation A2 in both clockwise and counter-clockwise rotational directions. As illustrated in FIG. 3, the second axis motor 34 is located within the first arm segment 15 of arm 14, wherein motor 34 operatively connects to the patient restraint by a suitable intermediate structure 34A. Examples of intermediate structure 34A include, but are not limited to, chains, belts, gears, drive shafts, or the like and any combinations thereof. Alternately, the second axis motor 34 could also be directly connected to patient restraint 18 in a direct drive relation. In this configuration, second axis motor 34 may be located in other locations, such as within patient restraint 18.

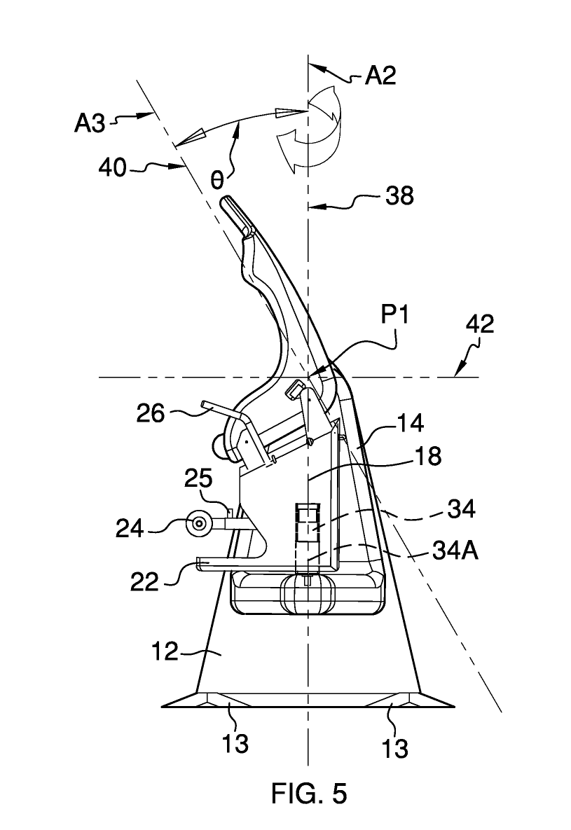

In embodiments, the patient restraint 18 can be in the form of a seat 20, which receives and restrains a patient against movement such that the patient is properly positioned in relation to the first axis of rotation A1 and the second axis of rotation A2. In this manner, a patient is received and restrained by the patient restraint 18 such that the first axis of rotation A1 and the second axis of rotation A2 intersect at approximately the center of gravity of the patient at P1. Alternatively, P1 may be located relative to a particular organ that is being treated, such as, for example the center of mass of an organ. Further, the patient is received and restrained by the patient restraint 18 such that the first axis of rotation A1 is parallel to the transverse plane 42 of the patient and normal to the sagittal plane 38 of the patient, and such that the second axis of rotation A2 is parallel to the sagittal plane 38 of the patient and is rotated at a constant and fixed angle Ø relative to the coronal plane 40 of the patient, as best seen in FIG. 5.

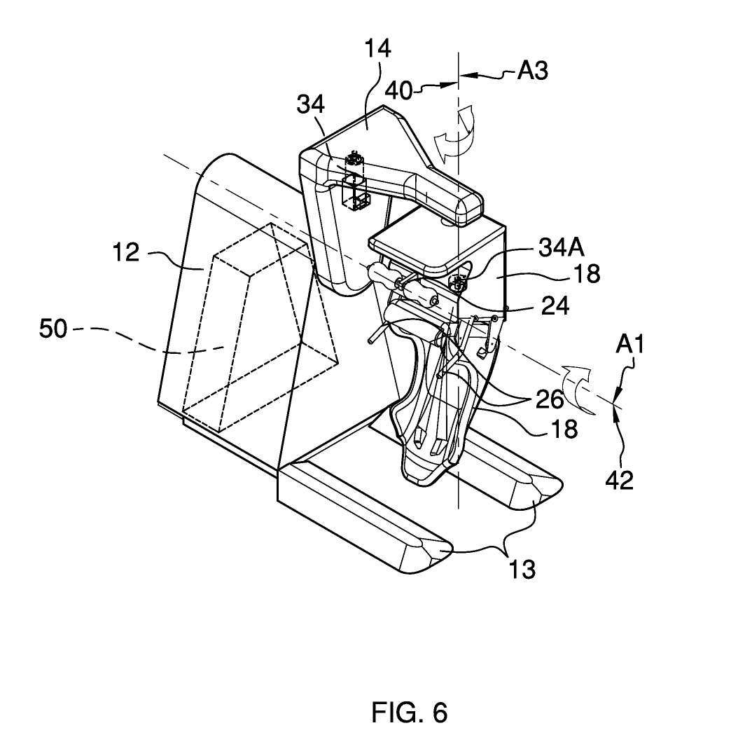

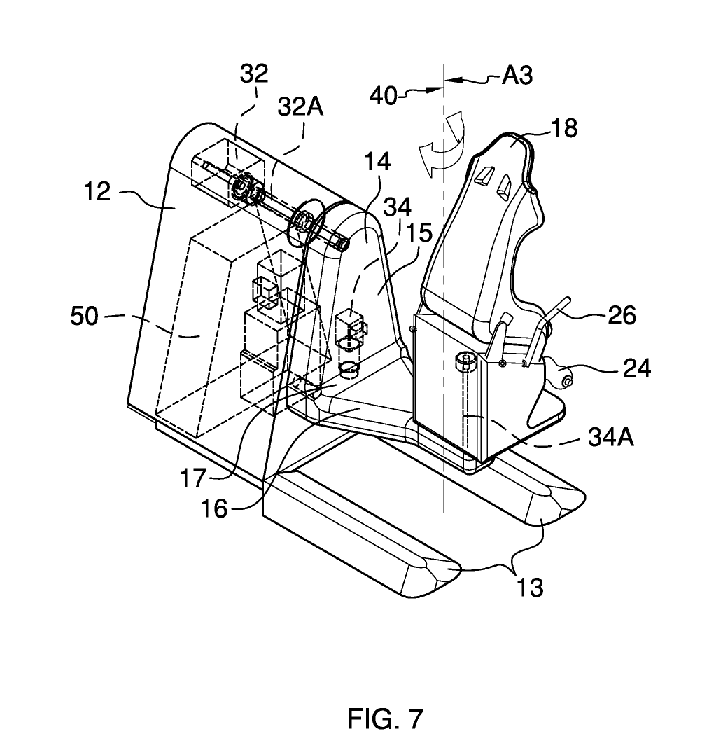

Arm 14 rotates about the first axis of rotation A1 360-degrees in both clockwise and counter-clockwise direction, as illustrated in FIG. 6. Similarly, patient restraint 18 rotates about the second axis of rotation A2 360-degrees in both clockwise and counter-clockwise direction, as illustrated in FIG. 7. By configuring the arm 14 and patient restraint 18 to rotate about first and second axes of rotation A1 and A2 respectively, the patient is able to rotate in relation to the three planes of the semicircular canals allowing specific isolation of each canal. These planes, orthoganol to each other, all of which should be familiar to a person of skill in the art.

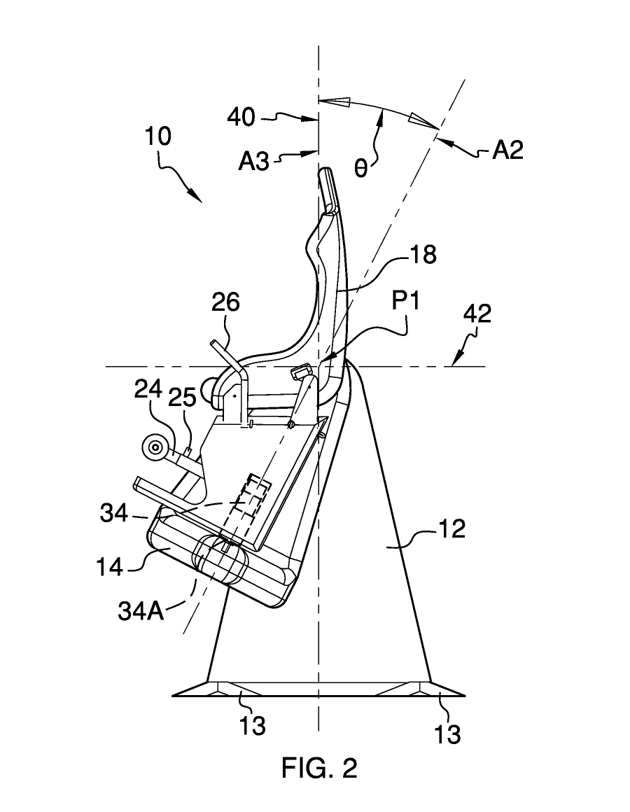

The second axis of rotation A2 is orthogonal to the first axis of rotation A1. Patient restraint 18 supports the patient at a third axis A3, which is parallel with the patient sagittal 38 plane. The first axis of rotation A1, second axis of rotation A2, and third axis A3 intersect at a point P1, which may be approximately equal to the patient's center of gravity. Centering rotation about the patient's center of gravity reduces the force required to manipulate the patient, thus increasing the efficiency and stability of apparatus 10. The relationship between axes A1 and A2 is best illustrated in FIG. 3. Similarly, the relationship between axes A2 and A3 is best illustrated in FIGS. 2 & 5.

The orthogonal configuration of first axis of rotation A1 and second axis of rotation A2 allows for the proper alignment of the semi-circular canals undergoing diagnosis or treatment. With proper alignment, an operator is able to quickly diagnose and treat conditions related to abnormalities in the semi-circular canals. In one embodiment, proper alignment is provided by an approximately 30-degree offset of the patient restraint 18 from the second axis of rotation A2 in the forward direction in the sagittal plane 38, positioning the patient relative to the third axis A3, as shown in FIG. 5. The addition of the 30-degree offset third axis A3 allows greater accuracy with alignment of the semi-circular canals to be isolated and the spatial position required for the diagnosis or treatment procedure. With this configuration, the patient still rotates about the second axis of rotation A2, but the patient is positioned within the patient restraint 18 such that the patient faces forward 30-degrees, instead of being positioned parallel to the second axis of rotation A2. Although rotating about only two axes of rotation, the operator is able to rotate the patient about the three planes in which the semicircular canals are orientated. With this capability, the operator can rotate a patient about a plane, which aligns with the specific semi-circular canal the operator wishes to diagnose or treat, without requiring excessive complexity or machinery.

Additionally, the patient restraint 18 relative to axis A3 may rotate at point P1 to become parallel to the second axis of rotation A2. With this capability, the operator can rotate a patient in relation to the three primary planes of the human body. These planes include the sagittal plane 38, the coronal plane 40, and the transverse plane 42, all of which should be familiar to a person of skill in the art. Although rotating about only two axes of rotation, the operator is able to rotate the patient about all three orthogonal body planes.

The ability to isolate each semi-circular canal using only two axes of rotation allows the operating dimensions of apparatus 10 to be considerably smaller than competing devices with comparable functionality, such as a circular or spherical truss device. First, this is because apparatus 10 does not require additional space and structural support to rotate a patient about a third axis of rotation. Second, apparatus 10 does not require a fixed frame about the circumference of the device, as otherwise required to allow rotation about the corresponding axis of rotation for devices in the prior art.

Additionally, in the clinical setting, apparatus 10 allows the operator to rotate the patient about all dimensions without requiring the patient to manually position the patient's head or neck. This capability of rotating a patient without requiring head or neck movement allows the operator to examine and diagnose patients with neck or back injuries. These injured patients would otherwise be prohibited from receiving medical assistance, due to their inability to position themselves as required by manual examination. In addition, the fully suspended patient can undergo the prescribed maneuvers in a more rapid and precise manner.

The patient restraint 18 can include a foot platform 22, leg restraint 24, handles 26, a head restraint 27, and a torso restraint 28. In embodiments, the foot platform 22 would be located near the bottom of patient restraint 18. As further illustrated in FIGS. 3-5, the inclusion of a leg restraint 24 can provide an extension of patient restraint 18, capable of being detached from patient restraint 18 while loading and unloading patients. Leg restraint 24 optionally has horizontal extensions 23 that allow apparatus 10 to securely hold a patient's legs during operation. To detach the leg restraint 24, the operator would lift a locking leg restraint pin 25 (shown in FIGS. 2&5) that allows the leg restraint 24 to be temporarily detached from patient restraint 18. After the patient is positioned into the patient restraint 18, the operator can reattach the leg restraint 24 by lifting the leg restraint pin 25 and reinserting leg restraint 24 into patient restraint 18. A person of skill in the art will realize that variations to this configuration exist that remains within the scope and spirit of the invention.

In embodiments, handles 26 attach to patient restraint 18 to provide the patient with additional support and security. Handles 26 can be mounted at approximately 100-degrees offset from the neutral position of the wrist inline with the axis of the forearm, providing maximum ergonomic comfort for the patient. A person skilled in the art will realize that the handles 26, if included, can be set at different angles without affecting the functional operation of apparatus 10.

A person skilled in the art will also realize torso restraint 28 may include any number of fastening straps 29 required to secure the patient for movement with the patient restrain 18. Fastening straps 29 may be provided in the form of a 5-point harness as illustrated. A person of skill in the art will realize other fastening methods may be used within the spirit and scope of the invention, such as but not limited to a rigid harness or an over-the-shoulder brace. In addition to leg restraint 24 and torso restraint 28, apparatus 10 may also include a head restraint 27. Head restraint 27 is provided to secure the patient's head to the patient restraint 18 and ensure conjoint movement between the patient's head and the patient restraint 18. The head restraint 27 also prevents the patient's head from receiving any shock or unexpected motions during the operation of apparatus 10.

In an embodiment the first axis motor 32 and the second axis motor 34 are pulse-width-modulation motors. The first axis motor 32 and the second axis motor 34 may be provided with an error sensing feedback system. Such error feedback systems are well known in the art, and require no description herein. Additionally, the first axis motor 32 and second axis motor 34 may passes through a slip ring 36, operatively connecting base 12 to arm 14. Slip ring 36 allows control signals 52 from controller 50 and power transmission to conduct through arm 14 to the second axis motor 34, which controls the rotation of patient restraint 18. An additional slip ring 36B could also be located between arm 14 and patient restraint 18 as required.

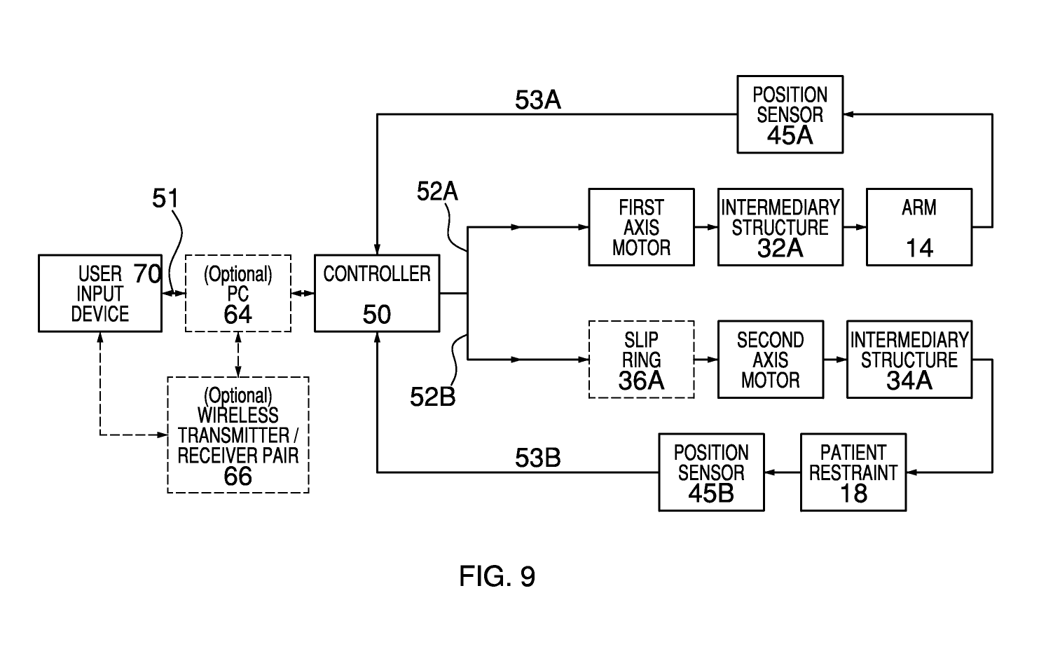

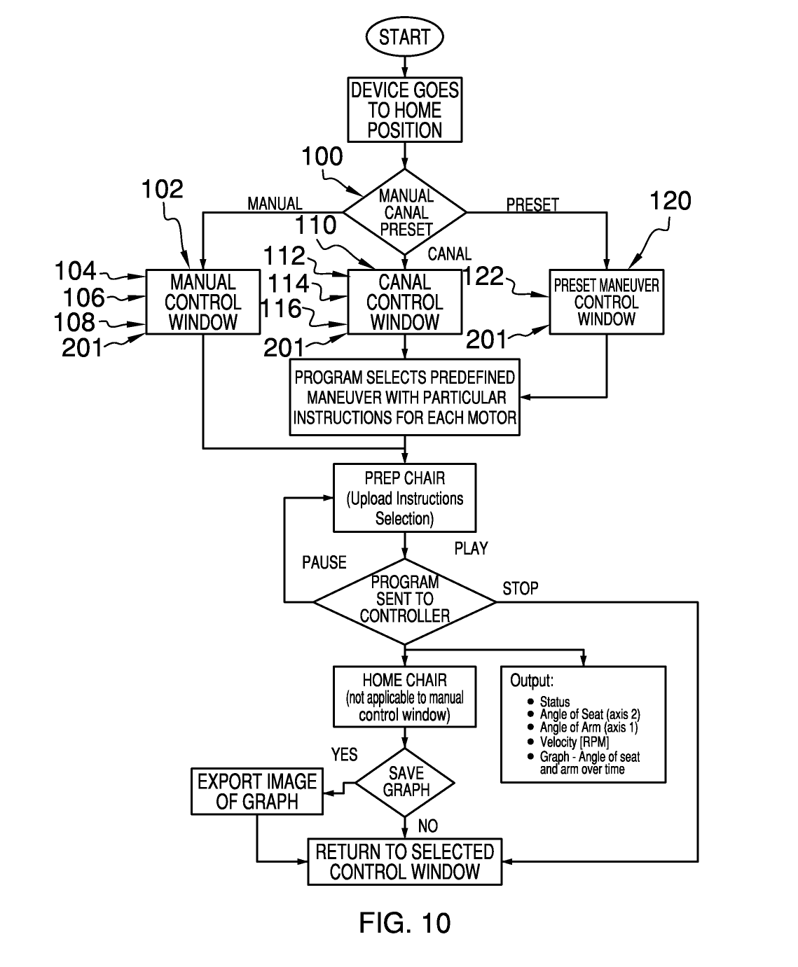

As previously mentioned, apparatus 10 includes a controller 50, which is capable of transmitting control signals 52 to the first axis motor 32 (via control signal 52A) and second axis motor 34 (via control signal for 52B), directing the rotation for arm 14 and patient restraint 18, respectively. Similarly, controller 50 could receive feedback signals 53 from the first axis motor 32 (via feedback signal 53A) or second axis motor 34 (via feedback signal for 53B), reporting error feedback. FIG. 9 illustrates how controller 50 can connect to and operate first axis motor 32 and second axis motor 34. Also shown in FIG. 9, a user input device 70 may be included to transmit or receive command signals 51 with controller 50. FIG. 10 further illustrates control methods that may include options such as manual control, canal specific control, or preset programs. A person of skill in the art will realize that other methods of control may exist that are consistent with the nature of this invention.

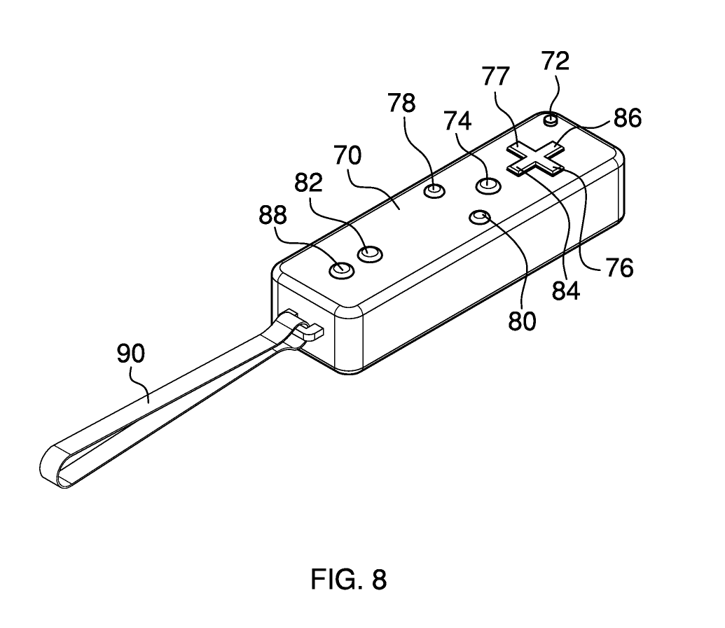

The operator controls the motions of apparatus 10 by providing command signals 51 that are received by controller 50. A person of skill in the art will realize a variety of methods to interface with controller 50, which would be encompassed within the scope of the invention. For example, one method of interfacing with controller 50 involves an operator directly controlling apparatus 10 through an interface attached to body 12. A second example could include a voice control configuration. A third embodiment can include a computer 64 attached to apparatus 10. In this embodiment, the programming and control of apparatus 10 would be performed on computer 64, which would then transmit the electronic instructions to apparatus 10 through an operable means known to a person of skill in the art. In yet another embodiment, the operator would control apparatus 10 through a user interface device 70, as shown in FIG. 8. These examples are not meant to be exhaustive, as other similar control methods exist that would be within the scope of the invention.

Referring to FIG. 8, and as previously mentioned, apparatus 10 may further include a user input device 70 used to communicate with the controller 50. The addition of a user input device 70 allows the operator to focus on the patient and the diagnosis/treatment taking place, since the operator is not required to monitor a control interface that may require extra attention. This user input device 70 may contain a multitude of user inputs, such as buttons. In an illustrative configuration, user input device 70 may include user inputs 72, 74, 76, 80, 82, 84, and 86. A person of skill in the art will realize that the user input device 70 may be of various forms and/or contain various types of input mechanisms, and still fall within the scope and spirit of the invention. In addition to user inputs, the user input device 70 optionally includes a status light 88 to give the operator visual feedback and/or a wrist strap 90 to resist accidents due to the user input device 70 leaving the operator's hand. Also, the user interface device 70 may be connected to a controller 50 through an optional intermediary computer 64. User input device 70 may communicate with controller 50 through a wired or wireless connection of a type known to a person of skill in the art. If a wireless connection is used, the user input device 70 and controller 50 will require a receiver/transmitter pair 66. Examples of a receiver/transmitter pair 66 include, but are not limited to, a wireless radio or infrared communications device. If an intermediary computer 64 is wirelessly connected, the computer 64 will require a receiver/transmitter pair 66, potentially replacing the requirement of the receiver/transmitter pair 66 in controller 50.

As previously mentioned, in one embodiment a user input device 70 connects to a computer 64 wirelessly. In order for the computer 64 to receive and transmit wireless data with the user input device 70, the computer 64 will require a receiver/transmitter pair 66. A person skilled in the art will recognize that if the receiver/transmitter pair 66 contains a wireless radio, it will also contain an antenna designed appropriately to transmit and receive wireless signals at the appropriate frequency and the control logic necessary to communicate with the user input device 70. The wireless connection could be established via either a proprietary wireless protocol or an open standard, such as IEEE 802.11 Wi-Fi or IEEE 802.15.1 Bluetooth. One embodiment connects wirelessly within the 2.4 to 2.5 GHz spectrum band, but a person skilled in the art would recognize that a wireless connection could be established in any open or licensed frequency range. In order to keep costs relatively low, the wireless link between the user input device 70 and computer 64 would benefit from connecting through an open wireless protocol.

In an alternate embodiment, the user input device 70 could be connected to controller 50 or computer 64 through a wired connection and protocol capable of transmitting a signal between the two devices. As an example, a Universal Serial Bus (USB) connection could be used for wired communication. A person skilled in the art will recognize other protocols and connections capable of providing a means of communication between the user input device 70, controller 50, and/or computer 64 within the scope of this invention.

In operation, it now can be understood that apparatus 10 facilitates the effective diagnosis and treatment of a patients conditions arising from abnormalities with the vestibular organs. This effectiveness is due to the manner in which apparatus 10 is capable of rotating a patient about the same plane as the patient's semi-circular canal corresponding with the particular abnormality of the vestibular organs. Also, since apparatus 10 is capable of rotating a patient about all three necessary body planes and planes of the semicircular canals with rotation about two axes, apparatus 10 provides an operator with an excellent tool for diagnosis and treatment of patients without the burdensome requirement of excessive space or unnecessary complexity. In addition, the fully suspended patient can undergo the prescribed maneuvers in a more rapid and precise manner.

To treat a patient using apparatus 10, an operator will load a patient into the patient restraint 18. To ensure safety during operation of apparatus 10, the patient will be secured to patient restraint 18 via a torso restraint 28, and optionally via a leg restraint 24 and/or a head restraint 27. Once the patient has been securely fastened into the patient restraint, the operator may begin using apparatus 10 to diagnose or treat the patient conditions.

Apparatus 10 may be pre-programmed with a set of tests for diagnosis and treatment of patients with vestibular organ abnormalities. After an initial configuration of the controller 50, the operator may quickly and efficiently run diagnostic tests on a patient without requiring the patient to comprehend the operator's instructions. Hence, the operator does not waste time correcting a patient that misunderstands the operator's instruction. Also, due to the nature of diagnosing conditions caused by an abnormality of the vestibular organs, the use of apparatus 10 allows the operator to efficiently diagnose and treat the patient without having to continually compensate for patient awareness of the tests performed.

With the inclusion of a user input device 70, the operator may elect to control apparatus 10 via the device 70. By using the user input device 70, the operator is able to keep his focus on the patient since he or she is not required to interact with apparatus 10 through an interface located elsewhere, such as on apparatus 10 or on a computer 64. The enhanced communication provided by the operator's increased focus, since using a user input device 70 allows the operator to focus on the patient, facilitates greater awareness of the patient's responses and reactions to the diagnosis or treatment being performed with apparatus 10.

To operate apparatus 10 via the user input device 70, the operator will be able to press user inputs, such as buttons, on the surface of the user input device 70 which will be mapped to corresponding functions contained within the controller 50. In the following embodiment, the user inputs are buttons and assigned functions. A person skilled in the art will recognize that the user inputs may be configured differently and maintain the same operable functionality. Input or button assignments could be as such: 72, power on the user input device 70; 74, activate or pause the program loaded into the controller 50 of apparatus 10; 76, rotate the device counter-clockwise under manual operation; 77, rotate the device clockwise under manual operation; 78, decrease speed of rotation under manual operation; 80, increase speed of rotation under manual operation; 82, stop or reset apparatus 10 to the home or zero position; 84, select arm 14 rotation direction; and 86 select chair rotation direction. The button assignments of this embodiment are given for illustrative purposes, and a person of skill in the art will recognize that user inputs and functions may be added or deleted without changing the nature and functionality of the user input device 70.

If the operator is controlling apparatus 10 through an intermediary computer 64, the operator will connect apparatus 10 to a computer interface program 68 running on the computer 64. Through use of the computer interface program 68, the operator may control apparatus 10 manually or upload a predetermined program.

A person of skill in the art will recognize the following embodiment is not exclusive, as variations in the computer interface program 68 may exist within the scope of the invention. In this embodiment, the operator will select between three primary modes of operation: manual operation, canal control, or preset maneuver control. Besides a persistent home chair input 201, which returns apparatus 10 to the home or zero position, the three modes of operation function exclusive of each other. For manual operation of apparatus 10, the operator will select manual control from a function list 100. The corresponding manual control window 102 will contain selectors for patient restraint 18 controls and arm 14 controls, as well as a rotation velocity selector 108. Through manipulating the patient restraint 18 controls 104, the operator can select whether to rotate the patient restraint 18 in the clockwise or counter-clockwise direction. Similarly, the operator can select whether to operate the arm 14 in the clockwise or counter-clockwise direction by adjusting the arm controls 106. Both arm 14 and patient restraint 18 controls can be set to clockwise, counter-clockwise, or off (no rotation). In addition, the manual control window 102 features a rotation velocity selector 108, which controls the velocity at which the selected arm 14 or patient restraint 18 rotates. After the operator selects his or her state of manual operation, he or she will instruct the computer interface program 68 to upload the instructions to apparatus 10.

Also, to diagnose or treat a specific semi-circular canal, the operator will select canal control from the function list 100. On the canal control window 110, the operator can use the canal selector 112 menu to select whether to focus diagnosis or treatment on the anterior, posterior, or horizontal semi-circular canal. The operator can also use a left/right selector 114 to select whether the left or right side of the patient will undergo diagnosis or treatment. Finally, the operator may use a diagnosis/treatment selector 116 to choose between diagnosing and treating the patient. After the operator selects a specific canal, and whether to diagnose or treat the selected canal, the operator will instruct the computer interface program 68 to upload the instructions to apparatus 10.

Finally, in this embodiment, the operator could select the preset maneuver control option from the function list 100. On the preset maneuver control window 120, the operator is presented with a list of presets 122, containing an assortment of preconfigured maneuvers. Such maneuvers may include, but are not limited to, Brandt's exercise, Dix Hallpike exercise, Semont exercise, or the Epply exercise. The operator would then instruct the computer interface program 68 to upload the instructions to apparatus 10. A person who is skilled in the art will be aware that other maneuvers can be programmed into the computer interface program 68. Also, a person who is skilled in the art will recognize organizational and syntax variations could exist within the implementation of the computer interface program 68, and the design and operation of the computer interface program 68 should not be confined to this embodiment.

After the selected mode of operation has been uploaded to the controller 50, the program monitors inputs entered by the user and manipulates apparatus 10 accordingly. During operation, computer interface program 68 generates a variety of outputs, such as status, time, angle of seat, angle of arm, and rotational velocity. In one embodiment, computer interface program 68 also generates a graph that visually represents two output signals, such as angle of seat/arm versus time. Before computer interface program 68 completes operation, the user will be provided with an option to save the output graph. After optionally saving the graph, and completion of all other operations, the computer interface program 68 returns user to the selected control window to await a user input selecting a new mode of operation.

It is intended that any alternative embodiments of the present invention that may result from changes in the application or method of operation, manufacture, shape, size, material, or use that have not been explicitly characterized or described in the foregoing written description be considered obvious to a person skilled in the art, and remain within the scope of the invention.

Although the present invention has been described in detail with reference to the foregoing embodiments, a person of skill in the art will recognize that changes in form, style, and detail may be made without departing from the spirit and scope of the invention claimed in this patent application. It will be understood that various modifications may be made without departing from the spirit and scope of the invention. Accordingly, other embodiments are within the scope of the following claims.

What is claimed is:

1. A patient spatial movement apparatus for moving a patient through space, the patient spatial movement apparatus comprising:
a base;
an arm attached to and supported by said base for rotation about a first axis of rotation;
a patient restraint attached to and supported by said arm for rotation about a second axis of rotation;
said patient restraint receiving a patient such that said second axis of rotation is at a constant and fixed angle relative to the transverse plane of the patient;
wherein said first axis of rotation is normal to the sagittal plane of the patient; and
wherein said first axis of rotation is orthogonal to said second axis of rotation.

2. The patient spatial movement apparatus of claim 1, wherein said first axis of rotation and said second axis of rotation intersect about the center of gravity of a patient received by said patient restraint.

3. The patient spatial movement apparatus of claim 1, wherein said arm comprises:
a first arm segment having opposed first and second ends;
a second arm segment having opposed first and second ends;
said first arm segment attached to said base at said first end thereof;
said second arm segment attached at said first end thereof to said second end of said first arm segment and extending outwardly therefrom; and
said patient restraint attached to said second arm segment.

4. The patient spatial movement apparatus of claim 1, wherein said constant and fixed angle is about 30-degrees.

5. The patient spatial movement apparatus of claim 3, wherein said second arm segment extends parallel to said first axis of rotation, and wherein said first arm segment extends normal to said first axis of rotation.

6. The patient spatial movement apparatus of claim 1, further comprising:
a controller;
a first motor operatively connected to said arm and operable to rotate said arm about said first axis of rotation;
second motor operatively connected to said patient restraint and operable to rotate said patient restraint about said second axis of rotation;
said first motor and second motor connected to said controller to receive control signals therefrom to effect the operation of said first motor to rotate said arm about said first axis of rotation and to effect the operation of said second motor to rotate said patient restraint about said second axis of rotation.

7. The patient spatial movement apparatus of claim 6, wherein said controller operates to receive command signals and based upon said command signals effects the operation of said first motor and said second motor.

8. The patient spatial movement apparatus of claim 7, further comprising:
a user input device, said user input device generating said command signals.

9. The patient spatial movement apparatus of claim 8, wherein said user input is wirelessly connected to said controller.

10. The patient spatial movement apparatus of claim 6, wherein said controller is programmed with one or more procedures for controlling the operation of said first motor and said second motor to effect movement of a patient received by said patient restraint according to a predefined sequence of movements.

11. The patient spatial movement apparatus of claim 1, wherein said patient restraint is a seat.

12. The patient spatial movement apparatus of claim 10, further comprising:
a body restraint, wherein said torso restraint is operative with said seat to restrain a patient received by said seat against movement.

13. The patient spatial movement apparatus of claim 11, wherein said body restraint comprises:
a torso restraint;
a head restraint; and
a leg restraint.
